(19) United States
(12) Patent Application Publication
(10) Pub. No.: US 2010/0204486 A1
(43) Pub. Date: Aug. 12, 2010

(54) Process for the Synthesis of (+)and (-)-1 Aryl-3-Azabicyclo(3.1.0) Hexanes

(75) Inventors: Edward G. Corley, Freehold, NJ (US); Jerry A. Murry, Rahway, NJ (US); Bryon Simmons, Princeton, NJ (US); Feng Xu, Staten Island, NY (US)

Correspondence Address: MERCK, P O BOX 2000, RAHWAY, NJ, 07065-0907, US

(73) Assignee: MERCK & CO., INC., Rahway, NJ (US)

(21) Appl. No.: 12/226,801

(22) PCT Filed: Apr. 27, 2007
(86) PCT No.: PCT/US2007/010324

§ 371 Date: Oct. 27, 2008

Related U.S. Application Data
(60) Provisional application No. 60/796,086, filed on Apr. 28, 2006.

(51) Int. Cl. C07D 209/52; C07C 255/36; C07C 211/37; C07C 211/35

(52) U.S. Cl. 548/452; 558/410; 564/307

(57) ABSTRACT

The present invention is concerned with novel processes for the preparation of (+)-1-aryl-3-azabicyclo[3,10]hexanes or a pharmaceutically acceptable salt thereof, and (−)-1-aryl-3-azabicyclo[3,10]hexanes or a pharmaceutically acceptable salt thereof. These compounds have pharmaceutical utility and are known to be useful for treating e.g., pain, depression, anxiety disorders, eating disorders and urinary incontinence.

### BACKGROUND OF THE INVENTION

The present invention relates to processes for the preparation of (+)-1-aryl-3-azabicyclo[3.1.0]hexanes or a pharmaceutically acceptable salt thereof, and (−)-1-aryl-3-azabicyclo[3.1.0]hexanes or a pharmaceutically acceptable salt thereof. In one embodiment, the present invention relates to processes for the preparation of (+)-1-(4-methylphenyl)-3-azabicyclo[3.1.0]hexane, or a pharmaceutically acceptable salt thereof, and (−)-1-(4-methylphenyl)-3-azabicyclo[3.1.0]hexane or a pharmaceutically acceptable salt thereof. These compounds are known to be useful for treating e.g., pain, depression, anxiety disorders, eating disorders and urinary incontinence (see U.S. Pat. Nos. 4,231,935 and 4,196,120 and J. Med. Chem., 1981, 24, 481).

The general processes disclosed in the art for the preparation of racemic, (+) and (−)-1-aryl-3-azabicyclo[3.1.0]hexane result in relatively low and inconsistent yields of the desired product (see e.g., U.S. Pat. Nos. 4,118,417, 4,131,611, 4,196,120, 4,231,935, and 4,435,419, PCT Publications WO 2004/012722 and WO 2004/043920, and Epstein, et al., J. Med. Chem., 1981, 24, 481). Some of such processes rely on the use of expensive reagents. In contrast to the previously known processes, the present invention provides effective methodology for the preparation of (+) or (−)-1-aryl-3-azabicyclo[3.1.0]hexanes in relatively high yield and enantiomeric purity. It will be appreciated that (+) or (−)-1-aryl-3-azabicyclo[3.1.0]hexanes, including (+) and (−)-1-(4-methylphenyl)-3-azabicyclo[3.1.0]-hexane are useful therapeutic agents. As such, there is a need for the development of processes for the preparation of (+) and (−)-1-aryl-3-azabicyclo[3.1.0]hexanes which are readily amenable to scale-up, use cost-effective and readily available reagents, and which are therefore capable of practical application to large scale manufacture. Accordingly, the subject invention provides a process for the preparation of (+)-1-aryl-3-azabicyclo[3.1.0]hexanes, (−)-1-aryl-3-azabicyclo[3.1.0]hexanes, including (+)-1-(4-methylphenyl)-3-azabicyclo[3.1.0]hexane and (−)-1-(4-methylphenyl)-3-azabicyclo[3.1.0]hexane via a very simple, short and highly efficient synthesis.

### SUMMARY OF THE INVENTION

The present invention provides novel processes for the preparation of a compound of the formula I:

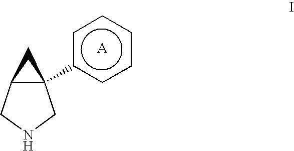

or a pharmaceutically acceptable salt thereof,
wherein the ring A is phenyl, which is unsubstituted or substituted with one or more of a group which is selected from the group consisting of: bromo, chloro, fluoro, iodo, —NO₂, —CN, —NH₂, —NH(C₁₋₈alkyl), —N(C₁₋₈alkyl)₂, —O—C₁₋₈alkyl, C₁₋₈alkyl, C₂₋₈alkenyl, C₂₋₈alkynyl, C₃₋₈cycloalkyl, and trifluoromethyl, with the proviso that A is not 3,4-dichlorophenyl, or a compound of the formula Ib:

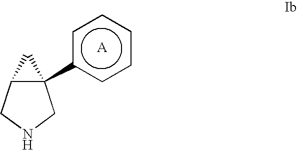

or a pharmaceutically acceptable salt thereof.

### DETAILED DESCRIPTION OF THE INVENTION

The present invention provides novel processes for preparing a compound of the formula I:

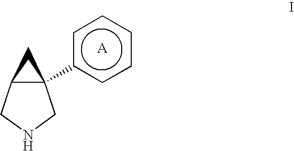

or a pharmaceutically acceptable salt thereof,
wherein the ring A is phenyl, which is unsubstituted or substituted with one or more of a group which is selected from the group consisting of: bromo, chloro, fluoro, iodo, —NO₂, —CN, —NH₂, —NH(C₁₋₈alkyl), —N(C₁₋₈alkyl)₂, —O—C₁₋₈alkyl, C₁₋₈alkyl, C₂₋₈alkenyl, C₂₋₈alkynyl, C₃₋₈cycloalkyl, and trifluoromethyl, with the proviso that A is not 3,4-dichlorophenyl, comprising:
contacting a phenyl-acetonitrile and (S)-epichlorohydrin of the formula:

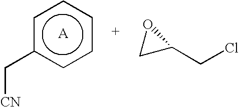

wherein the ring A is phenyl, which is unsubstituted or substituted with one or more of a group which is selected from the group consisting of: bromo, chloro, fluoro, iodo, —NO₂, —CN, —NH₂, —NH(C₁₋₈alkyl), —N(C₁₋₈alkyl)₂, —O—C₁₋₈alkyl, C₁₋₈alkyl, C₂₋₈alkenyl, C₂₋₈alkynyl, C₃₋₈cycloalkyl, and trifluoromethyl, with the proviso that A is not 3,4-dichlorophenyl,
in the presence of a base,
to give cyclopropyl compounds of the formula II:

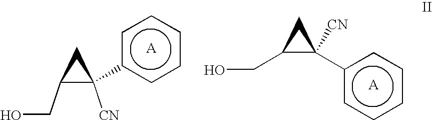

followed by reducing the compounds of formula II with a reducing agent to give amino alcohol compounds of the formula III:

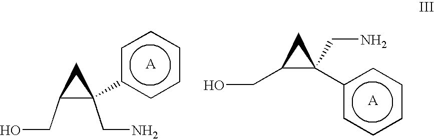

followed by chlorinating the compounds of formula III with a chlorinating agent to give chloro compounds of the formula IV:

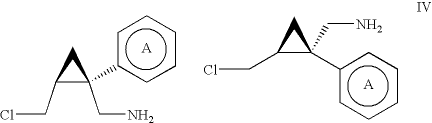

followed by cyclodehydration of the compounds of the formula IV with a base to give the compound of the formula I, or a pharmaceutically acceptable salt thereof.

The present invention is further directed to a process for preparing a compound of the formula Ib:

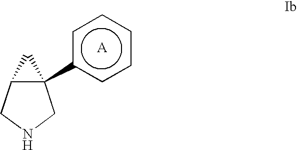

or a pharmaceutically acceptable salt thereof,
comprising:
contacting a phenyl-acetonitrile and (R)-epichlorohydrin of the formula:

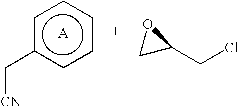

wherein the ring A is phenyl, which is unsubstituted or substituted with one or more of a group which is selected from the group consisting of: bromo, chloro, fluoro, iodo, —NO₂, —CN, —NH₂, —NH(C₁₋₈alkyl), —N(C₁₋₈alkyl)₂, —O—C₁₋₈alkyl, C₁₋₈alkyl, C₂₋₈alkenyl, C₂₋₈alkynyl, C₃₋₈cycloalkyl, and trifluoromethyl, with the proviso that A is not 3,4-dichlorophenyl,
in the presence of a base,
to give cyclopropyl compounds of the formula IIb:

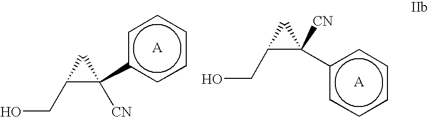

followed by reducing the compounds of formula IIb with a reducing agent to give amino alcohol compounds of the formula IIIb:

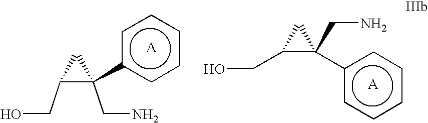

followed by chlorinating the compounds of formula IIIb with a chlorinating agent to give chloro compounds of the formula IVb:

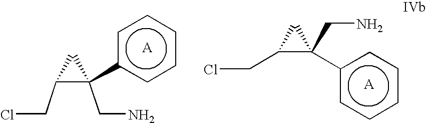

followed by cyclodehydration of the compounds of the formula IVb with a base to give the compound of the formula Ib, or a pharmaceutically acceptable salt thereof.

In an embodiment of the present invention, the ring A is phenyl, which is unsubstituted or substituted with one or more of a group which is selected from the group consisting of bromo, chloro, dimethylamino, ethyl, methoxy, methyl, and trifluoromethyl, with the proviso that A is not 3,4-dichlorophenyl.

In an embodiment of the present invention, the ring A is selected from the group consisting of:

(1) phenyl,

(2) 4-bromophenyl,

(3) 2-chlorophenyl,

(4) 3-chlorophenyl,

(5) 4-chlorophenyl,

(6) 2,6-dichlorophenyl,

(7) 4-(dimethylamino)phenyl,

(8) 2-methoxyphenyl,

(9) 4-methoxyphenyl,

(10) 4-methylphenyl,

(11) 3-trifluoromethylphenyl, and

(12) 4-trifluoromethylphenyl.

In an embodiment of the present invention, the ring A is 4-methylphenyl.

In one embodiment, the present invention is directed to a process for preparing a compound of the formula I-2:

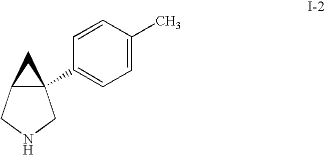

or a pharmaceutically acceptable salt thereof,
comprising:
contacting 4-methylphenylacetonitrile and (S)-epichlorohydrin of the formula:

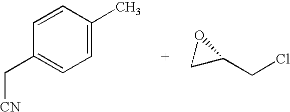

in the presence of a base,
to give cyclopropyl compounds of the formula II-2:

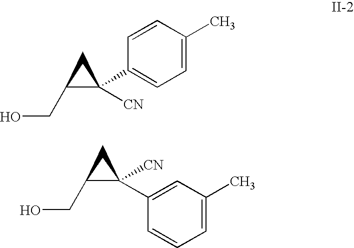

followed by reducing the compounds of formula II with a reducing agent to give amino alcohol compounds of the formula III:

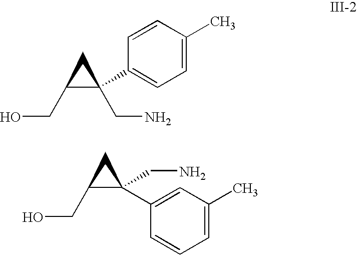

followed by chlorinating the compounds of formula III with a chlorinating agent to give chloro compounds of the formula IV:

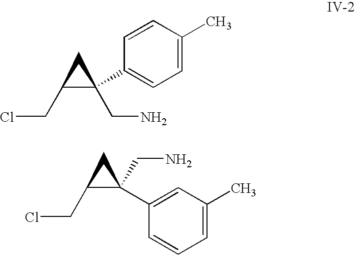

followed by cyclodehydration of the compounds of the formula IV with a base to give the compound of the formula I-2, or a pharmaceutically acceptable salt thereof.

The present invention is further directed to a process for preparing a compound of the formula I-2:

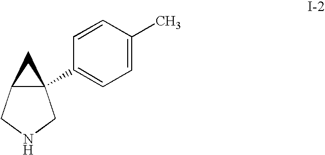

or a pharmaceutically acceptable salt thereof,
comprising:
cyclodehydration of the compound of the formula IV-2a:

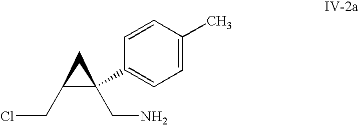

with a base to give the compound of the formula I-2, or a pharmaceutically acceptable salt thereof.

In an embodiment of the present invention the step of contacting the aryl-acetonitrile and (S)-epichlorohydrin [or (R)-epichlorohydrin] in the presence of a base to give cyclopropyl compounds of the formula II [or IIb], the base may be selected from sodium hexamethyldisilazide (NaHMDS), potassium hexamethyldisilazide (KHMDS), lithium hexamethyldisilazide (LiHMDS), potassium t-butoxide, potassium t-pentoxide, potassium amylate, lithium diisopropylamide (LDA), lithium tetramethylpiperidide (LiTMP), sec-butyllithium, or tert-butyllithium. Within this embodiment, the base is selected from sodium hexamethyldisilazide (NaHMDS), potassium hexamethyldisilazide (KHMDS) and lithium hexamethyldisilazide (LiHMDS). Further within this embodiment, the base is sodium hexamethyldisilazide (NaHMDS). Solvents for conducting the step of contacting the aryl-acetonitrile and (S)-epichlorohydrin [or (S)-epichlorohydrin] in the presence of a base to give cyclopropyl compounds of the formula II [or IIb] comprise an organic solvent. Within this embodiment, the organic solvent comprises toluene, tetrahydrofuran (THF), diethyl ether, diglyme, dimethoxyethane (DME), or methyl t-butyl ether. Further within this embodiment, the organic solvent is tetrahydrofuran. The step of contacting the aryl-acetonitrile and (S)-epichlorohydrin [or (S)-epichlorohydrin] in the presence of a base to give cyclopropyl compounds of the formula II [or IIb] is typically carried out at a temperature range of between about −30 and about 25° C. Within this embodiment, the temperature range is less than about 0° C. Further within this embodiment, the temperature range is between about −20 and about −5° C.

In an embodiment of the present invention the step of reducing of the compounds of formula II [or IIb] with a reducing agent to give amino alcohol compounds of the formula III [or IIIb], the reducing agent may be selected from borane dimethyl sulfide complex, borane tetrahydrofuran complex, sodium borohydride-borontrifluoride etherate, a diallylborane, 9-borabicyclo[3.3.1]nonane (9-BBN), and lithium alumium hydride (LAH). Further within this embodiment, the reducing agent is borane dimethyl sulfide complex. Solvents for conducting the step of reducing of the compounds of formula II with a reducing agent to give amino alcohol compounds of the formula III [or IIIb] comprise an organic solvent. Within this embodiment, the organic solvent comprises toluene, tetrahydrofuran (THF), diethyl ether, diglyme, dimethoxyethane (DME), or methyl t-butyl ether. Further within this embodiment, the organic solvent is tetrahydrofuran. The step of reducing of the compounds of formula II [or IIb] with a reducing agent to give amino alcohol compounds of the formula III [or IIIb] is typically carried out at a temperature range of between about −30 and about 25° C. Within this embodiment, the temperature range is less than about 0° C. Further within this embodiment, the temperature range is between about −20 and about −5° C.

In an embodiment of the present invention the step of chlorinating the compounds of formula III [or IIIb] with a chlorinating agent to give chloro compounds of the formula IV [or IV], the chlorinating agent may be selected from thionyl chloride, SO₂Cl₂, and Ph₃P/CCl₄. Further within this embodiment, the chlorinating agent is thionyl chloride. Solvents for conducting the step of chlorinating the compounds of formula III [or IIIb] with a chlorinating agent to give chloro compounds of the formula IV [or IVb] comprise an organic solvent. Within this embodiment, the organic solvent comprises toluene, tetrahydrofuran (THF), diethyl ether, diglyme, dimethoxyethane (DME), methyl t-butyl ether, ethyl acetate, isopropyl acetate or N-methylpyrrolidinone. Further within this embodiment, the organic solvent comprises tetrahydrofuran, dimethoxyethane and isopropyl acetate. The step of chlorinating the compounds of formula III [or IIIb] with a chlorinating agent to give chloro compounds of the formula IV [or IVb] is typically carried out at a temperature range of between about 0 and about 40° C. Within this embodiment, the temperature range is less than about 0° C. Further within this embodiment, the temperature is about 25° C.

In an embodiment of the present invention the step of cyclodehydration of the compounds of the formula I [or IVb] with a base to give the compound of formula [or Ib], the base may be selected from sodium hydroxide, potassium hydroxide, potassium bicarbonate, sodium bicarbonate, potassium carbonate, sodium carbonate, Et₃N, i-Pr₂NEt, DABCO, DBU, or other amine bases. Further within this embodiment, the base is sodium hydroxide. Solvents for conducting the step of cyclodehydration of the compounds of the formula IV [or IVb] with a base to give the compound of formula I [or Ib] comprise an aqueous solvent. In the step of cyclodehydration of the compounds of the formula IV [or IVb] with a base to give the compound of formula I [or Ib], the pH is typically at a range of between about 7-10. Within this embodiment, the pH is about 8-10. Further within this embodiment, the pH is about 8.5-9.5.

In an embodiment of the invention, the process steps are conducted sequentially without isolation of the intermediate compounds.

In a further embodiment, the present invention is directed to a process for the preparation of (+)-1-(4-methylphenyl)-3-azabicyclo[3.1.0]hexane as depicted below:

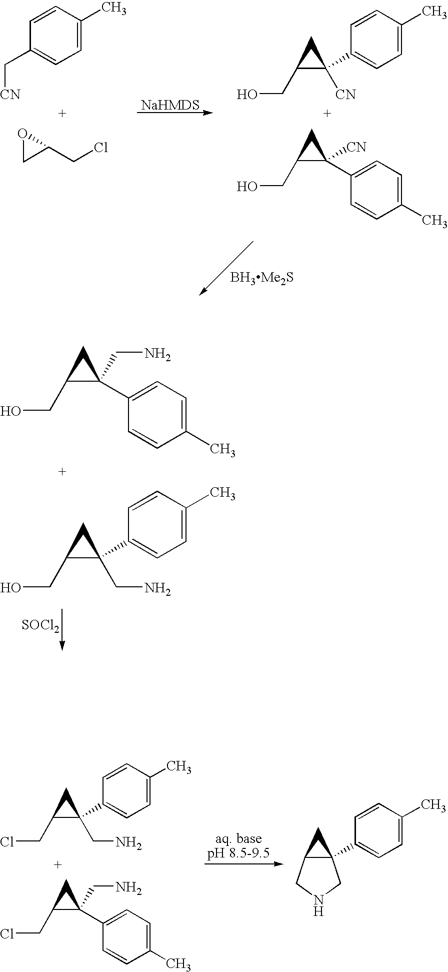

In a further embodiment, the present invention is directed to a process for the preparation of (+)-1-(4-methylphenyl)-3-azabicyclo[3.1.0]hexane as depicted below:

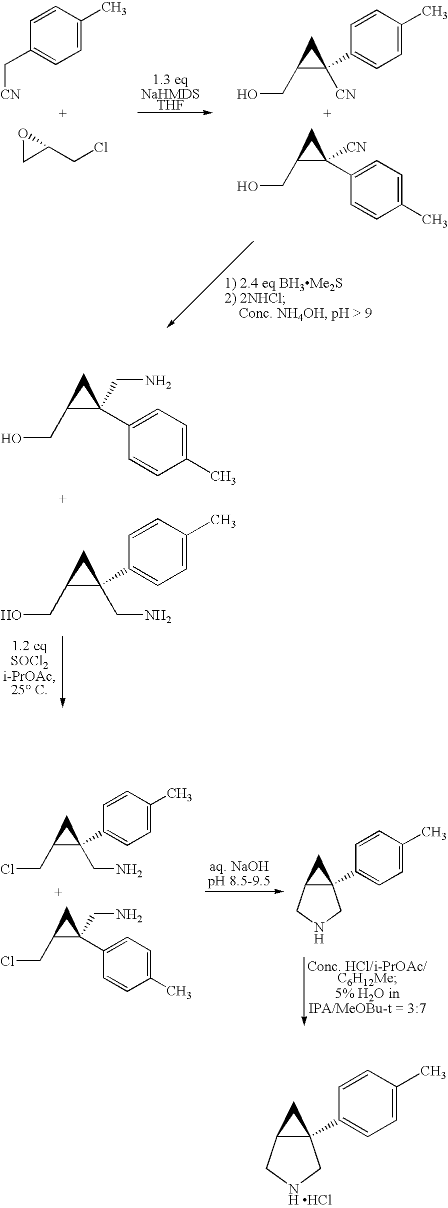

In an alternate embodiment, the present invention is directed to a compound which is selected from the group consisting of:

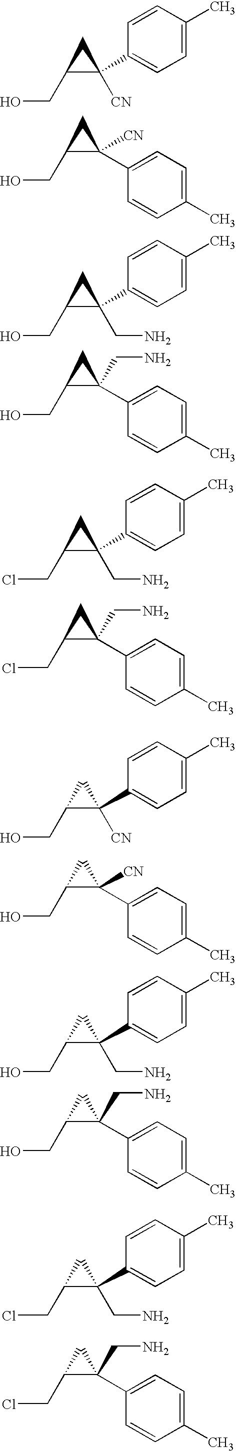

or a salt thereof.

The present invention provides a heavy metal-free synthesis that is efficient and atom economic so that (+)-1-(4-methylphenyl)-3-azabicyclo[3.1.0]hexane or (−)-1-(4-methylphenyl)-3-azabicyclo[3.1.0]hexane may be prepared via a single through process without requiring isolation of any intermediates. Starting from inexpensive, commercially available 4-methylphenylacetonitrile and (S)-epichlorohydrin (or (R)-epichlorohydrin), the key cyclopropane intermediate is constructed. Without further workup, the crude reaction mixture is reduced with borane dimethyl sulfide complex in one pot to afford the amino alcohol intermediates. The desired cis amino alcohol is directly cylodehydrated to give (+)-1-(4-methylphenyl)-3-azabicyclo[3.1.0]hexane or (−)-1-(4-methylphenyl)-3-azabicyclo[3.1.0]hexane. The whole synthesis may be conducted as a single stage through process to allow direct isolation of (+)-1-(4-methylphenyl)-3-azabicyclo[3.1.0]hexane HCl salt or (−)-1-(4-methylphenyl)-3-azabicyclo[3.1.0]-hexane HCl salt.

Another aspect of this invention is directed to the foregoing precesses wherein the (+)-1-(4-methylphenyl)-3-azabicyclo[3.1.0]hexane, or a pharmaceutically acceptable salt thereof, is present in an enantiomeric purity (enantiomeric excess) of greater than 90%, greater than 95%, greater than 98%, greater than 99%, greater than 99.5% (enantiomeric excess) or greater than 99.9% (enantiomeric excess).

Another aspect of this invention is directed to the foregoing precesses wherein the (−)-1-(4-methylphenyl)-3-azabicyclo[3.1.0]hexane, or a pharmaceutically acceptable salt thereof, is present in an enantiomeric purity (enantiomeric excess) of greater than 90%, greater than 95%, greater than 98%, greater than 99%, greater than 99.5% (enantiomeric excess) or greater than 99.9% (enantiomeric excess).

The term “pharmaceutically acceptable salts” refers to salts prepared from pharmaceutically acceptable non-toxic acids including inorganic or organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Specific acids include citric, hydrobromic, hydrochloric, maleic, phosphoric, sulfuric, fumaric, and tartaric acids. A specific acid is hydrochloric acid.

The present process is surprisingly efficient, minimizing the production of side products, and increasing productivity and purity. The starting materials and reagents for the subject processes are either commercially available or are known in the literature or may be prepared following literature methods described for analogous compounds. The skills required in carrying out the reaction and purification of the resulting reaction products are known to those in the art. Purification procedures include crystallization, distillation, normal phase or reverse phase chromatography.

The following examples are provided for the purpose of further illustration only and are not intended to be limitations on the disclosed invention. Unless otherwise noted, all reactions were conducted under N₂ atmosphere using standard air-free manipulation techniques. Solvents were purchased from Fisher Scientific Company and used without further purification. Commercial reagents were purchased either from Aldrich or Bayer and used without further purification. High performance liquid chromatography (HPLC) analysis was performed using Agilent Technology 1100 series instrument with ACE 5 C18 (240×4.6 mm I.D., 5 μm particle size) column. Proton nuclear magnetic resonance (¹H NMR) spectra were measured on Bruker Avance-400 instrument (400 MHz). Carbon nuclear magnetic resonance (¹³C NMR) spectra were measured on Bruker Avance-400 instrument (100 MHz) with complete proton decoupling. Chemical shifts are reported in ppm downfield from tetramethylsilane (TMS).

### Example 1

### (1R,5S)-(+)-1-(4-Methylphenyl)-3-azabicyclo[3,10]hexane[(+)-Bicifadine]

To a solution of 4-methylphenylacetonitrile (100 g) and S-(+)-epichlorohydrin (86.4 g) in MeOBu-t (800 mL) between −25 to −30° C. under atmosphere of N₂ was added LiHMDS (971 mL, 1M in hexane) dropwise over 2 h. After aging additional 2 h at −30° C., the reaction mixture was quenched into 5N HCl (448 mL) while the temperature was maintained below 20° C. After phase separation, the organic phase was washed with water (200 mL), azetropically dried and solvent-switched to DME (ca. 1 L) in vacuum. The above crude chlorohydrin in DME cooled to −15° C. NaHMDS solution (747 mL, 2M in THF) was added dropwise over 2 h. The reaction mixture was aged for additional 1 h between −15 to −20° C. BH₃-Me₂S (neat, 10M, 224 mL) was added dropwise over 1-2 h while the internal temperature was kept below 10° C. The reaction mixture was then gradually warmed to 40° C. over 2 h and aged for additional 3 h at 40° C. The reaction mixture was cooled to 20-25° C. and slowly quenched into a 4N HCl (934 mL) solution. The mixture was then aged for 1 h at 40° C. Ammonium hydroxide (401 mL) and i-PrOAc (500 mL) was added. The aqueous phase was back extracted with i-PrOAc (400 mL). The combined organic phase was washed with water (100 ml×2), azetropically dried and solvent-switched to i-PrOAc in vacuum (ca. 800 mL).

The above crude amino alcohol solution in i-PrOAc was slowly subsurface-added to a solution of SOCl₂ (0.911 mol, 66.5 mL) in i-PrOAc (450 mL) at ambient temperature over 2 h. After aging additional 1-3 h; 5.0 N NaOH (674 mL) was added over 1 h while the batch temperature was maintained at <30° C. with external cooling. The two-phase reaction mixture was stirred for 1 h at ambient temperature to allow pH to stabilize (usually to 8.5-9.0) with NaOH pH titration. The organic phase was washed with water (2×100 mL). Conc. HCl (68.5 mL) was added to the organic phases while the internal temperature was kept<30° C. The aqueous i-PrOAc was azeotropically concentrated in vacuum to a final volume of ca. 500 mL. Methylcyclohexane (350 mL) was added slowly over 2 h. The wet cake was displacement washed with 150 mL of 45% methylcyclohexan/1-PrOAc followed by a slurry wash (150 mL, i-PrOAc) and a displacement wash (150 mL, i-PrOAc). Typically isolated in yield: 65% over 4 steps, in >99 ee %.

(1R,5S)-(+)-1-(4-Methylphenyl)-3-azabicyclo[3,10]hexane HCl salt (100 g) was dissolved in a solution of i-PrOH (285 mL) and water (15 mL) at 75° C. Seeds (1 g) was added and the batch was allowed to cool to ambient temperature over 2-4 h. MeOBu-t (700 mL) was then added dropwise over 2 h. After aging 1 h at 20° C., the slurry was filtered. The wet cake was displacement washed with 150 mL of 30% i-PrOH in MeOBu-t followed by 2×150 mL 10% i-PrOH in MeOBu-t (slurry wash, then displacement wash). The (1R,5S)-(+)-1-(4-methylphenyl)-3-azabicyclo[3,10]hexane HCl salt was suction dried under N₂ at ambient temperature. Typical yield: 95%. ¹H-NMR (400 MHz, d₄-MeOH): δ 7.17 (m, 4H), 3.73 (d, J=11.4 Hz, 1H), 3.66 (dd, J=3.8, 11.4 Hz, 1H), 3.58 (d, J=11.4 Hz, 1H), 3.51 (d, J=11.4 Hz, 1H), 2.31 (s, 3H), 2.10 (m, 1H), 1.22 (t, J=7.5 Hz, 1H), 1.12 (t, J=5.4 Hz, 1H). ¹³C-NMR (100 MHz, d₄-MeOH): δ 138.3, 136.8, 130.6, 128.3, 52.3, 49.3, 32.3, 24.2, 21.2, 16.0. Anal. Calcd for C-₁₂H₁₆ClN: C, 68.73; H, 7.69; N, 6.68. Found: C, 68.52; H, 7.69; N, 6.64.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, reaction conditions other than the particular conditions as set forth herein above may be applicable as a consequence of variations in the reagents or methodology to prepare the compounds from the processes of the invention indicated above. Likewise, the specific reactivity of starting materials may vary according to and depending upon the particular substituents present or the conditions of manufacture, and such expected variations or differences in the results are contemplated in accordance with the objects and practices of the present invention. It is intended, therefore, that the invention be defined by the scope of the claims which follow and that such claims be interpreted as broadly as is reasonable.

1-17. (canceled)

18. A process for preparing a compound of the formula I: 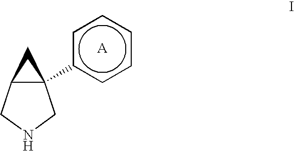
or a pharmaceutically acceptable salt thereof,
wherein the ring A is phenyl, which is unsubstituted or substituted with one or more of a group which is selected from the group consisting of: bromo, chloro, fluoro, iodo, —NO₂, —CN, —NH₂, —NH(C₁₋₈alkyl), —N(C₁₋₈alkyl)₂, —O—C₁₋₈alkyl, C₁₋₈alkyl, C₂₋₈alkenyl, C₂₋₈alkynyl, C₃₋₈cycloalkyl, and trifluoromethyl, with the proviso that A is not 3,4-dichlorophenyl,
comprising:
contacting a phenyl-acetonitrile and (S)-epichlorohydrin of the formula: 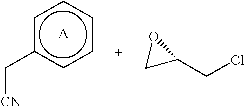
wherein the ring A is phenyl, which is unsubstituted or substituted with one or more of a group which is selected from the group consisting of: bromo, chloro, fluoro, iodo, —NO₂, —CN, —NH₂, —NH(C₁₋₈alkyl), —N(C₁₋₈alkyl)₂, —O—C₁₋₈alkyl, C₁₋₈alkyl, C₂₋₈alkenyl, C₂₋₈alkynyl, C₃₋₈cycloalkyl, and trifluoromethyl, with the proviso that A is not 3,4-dichlorophenyl,
in the presence of a base,
to give cyclopropyl compounds of the formula II: 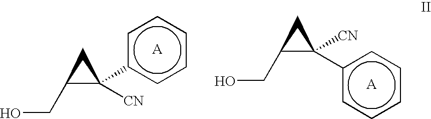
followed by reducing the compounds of formula II with a reducing agent to give amino alcohol compounds of the formula III: 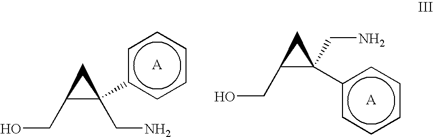
followed by chlorinating the compounds of formula III with a chlorinating agent to give chloro compounds of the formula IV: 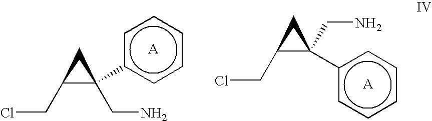
followed by cyclodehydration of the compounds of the formula IV with a base to give the compound of the formula I, or a pharmaceutically acceptable salt thereof.

19. A process for preparing a compound of the formula I-2: 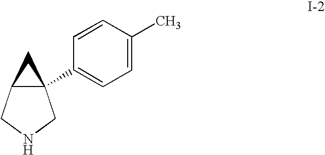
or a pharmaceutically acceptable salt thereof,
comprising:
cyclodehydration of the compound of the formula IV-2a: 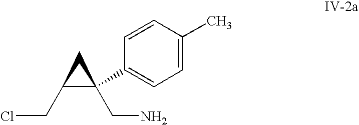
with a base to give the compound of the formula I-2, or a pharmaceutically acceptable salt thereof.

20. The process of claim 18 wherein the step of contacting the phenyl-acetonitrile and (S)-epichlorohydrin in the presence of a base to give cyclopropyl compounds of the formula II, the base is selected from sodium hexamethyldisilazide (NaHMDS), potassium hexamethyldisilazide (KHMDS), lithium hexamethyldisilazide (LiHMDS), potassium t-butoxide, potassium t-pentoxide, potassium amylate, lithium diisopropylamide (LDA), lithium tetramethylpiperidide (LiTMP), sec-butyllithium, and tert-butyllithium.

21. The process of claim 20 wherein the base is selected from sodium hexamethyldisilazide (NaHMDS), potassium hexamethyldisilazide (KHMDS) and lithium hexamethyldisilazide (LiHMDS).

22. The process of claim 21 wherein the base is sodium hexamethyldisilazide (NaHMDS).

23. The process of claim 18 wherein the step of reducing the compounds of the formula II, with a reducing agent to give amino alcohol compounds of the formula III, the reducing agent is selected from borane dimethyl sulfide complex, borane tetrahydrofuran complex, sodium borohydride-borontrifluoride etherate, a dialkylborane, 9-borabicyclo[3.3.1]nonane (9-BBN), and lithium alumium hydride (LAH).

24. The process of claim 23 wherein the reducing agent is borane dimethyl sulfide complex.

25. The process of claim 18 wherein the step of chlorinating the compounds of the formula III, with a chlorinating agent to give chloro compounds of the formula IV, the chlorinating agent is selected from thionyl chloride, SO₂Cl₂, and Ph₃P/CCl₄.

26. The process of claim 25 wherein the chlorinating agent is thionyl chloride.

27. The process of claim 18 wherein the step of cyclodehydration of the compounds of the formula IV, with a base to give the compound of formula I, the base is selected from sodium hydroxide, potassium hydroxide, potassium bicarbonate, sodium bicarbonate, potassium carbonate, sodium carbonate, Et₃N, i-Pr₂NEt, DABCO and DBU.

28. The process of claim 27 wherein the base is sodium hydroxide.

29. The process of claim 18 wherein the steps are conducted sequentially without isolation of the intermediate compounds.

30. The process of claim 18 wherein the ring A is phenyl, which is unsubstituted or substituted with one or more of a group which is selected from the group consisting of: bromo, chloro, dimethylamino, ethyl, methoxy, methyl, and trifluoromethyl, with the proviso that A is not 3,4-dichlorophenyl.

31. The process of claim 30 wherein the ring A is selected from the group consisting of:
(1) phenyl,
(2) 4-bromophenyl,
(3) 2-chlorophenyl,
(4) 3-chlorophenyl,
(5) 4-chlorophenyl,
(6) 2,6-dichlorophenyl,
(7) 4-(dimethylamino)phenyl,
(8) 2-methoxyphenyl,
(9) 4-methoxyphenyl,
(10) 4-methylphenyl,
(11) 3-trifluoromethylphenyl, and
(12) 4-trifluoromethylphenyl.

32. The process of claim 31 wherein the ring A is 4-methylphenyl.

33. A compound which is selected from the group consisting of: 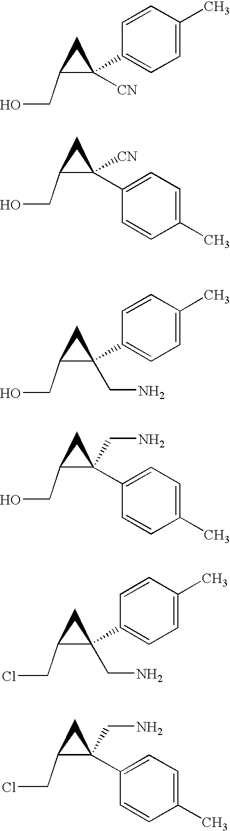
or a salt thereof.
